(19) United States
(12) Patent Application Publication
(10) Pub. No.: US 2021/0060557 A1
(43) Pub. Date: Mar. 4, 2021

(54) BIOLOGICAL DETECTION CARTRIDGE AND METHOD FOR PERFORMING THE SAME

(71) Applicant: Delta Electronics, Inc., Taoyuan City (TW)

(72) Inventors: Chi-Han Chiou, Taoyuan City (TW); Shu-Hsien Liao, Taoyuan City (TW); Ching-Yu Chang, Taoyuan City (TW)

(21) Appl. No.: 16/918,567

(22) Filed: Jul. 1, 2020

(30) Foreign Application Priority Data
Aug. 30, 2019 (CN) 201910815955.5

(51) Int. Cl. B01L 3/00; C12Q 1/18; C12M 1/34; C12M 1/00

(52) U.S. Cl. CPC B01L 3/502715; C12Q 1/18; B01L 2300/0829; C12M 41/14; C12M 41/46

(57) ABSTRACT

A biological detection cartridge includes a detection unit. The detection unit includes an incubation unit, a port, a ventilation structure and a cover. The incubation unit includes a culture well. The port is disposed adjacent to a first side of the incubation unit. The ventilation structure is disposed adjacent to a second side of the incubation unit. The ventilation structure includes a plurality of channels in communication with the incubation unit. The cover is disposed over the incubation unit and the ventilation structure.

### FIELD OF THE INVENTION

The present invention relates to a biological detection cartridge and a method for performing the same, and more particularly to a biological detection cartridge and a method for performing the same for an antimicrobial susceptibility test.

### BACKGROUND OF THE INVENTION

The existing standard antimicrobial susceptibility test is performed by using a 96-well plate. FIG. 1 schematically illustrates a 96-well plate for performing an antimicrobial susceptibility test. As shown in FIG. 1, the 96-well plate 1 comprises 96 wells W. A process of performing the antimicrobial susceptibility test will be described as follows. Firstly, antibiotic agent is placed into the wells W. Then, bacteria solution is placed into the wells W containing the antibiotic agent. After incubation for 16 to 20 hours, the bacteria growth is observed with naked eyes though the bottom side of the 96-well plate 1. Consequently, the drug resistance of the bacteria can be detected. This method is capable of testing a variety of drug susceptibility and strain tests and observing the test results with naked eyes. Due to these advantages, this method is gold-standard method of the antimicrobial susceptibility test.

However, this method still has some drawbacks. For example, since the antibiotic agent is serially diluted to form a concentration gradient, the sample-injecting procedure is complicated. Moreover, since only one lid is placed over the 96-well plate 1 to cover the openings, a cross-contamination problem is readily generated when the 96-well plate 1 is taken. Moreover, since the volume of the 96-well plate 1 and the volume of the sample drop (e.g., about 100˜150 μl) are large, the cost of waste disposal and the risk of contamination increase.

For overcoming the drawbacks of the conventional technologies, there is a need of providing an improved biological detection cartridge and an improved method for performing an antimicrobial susceptibility test while simplifying the sample-injecting procedure and avoiding the contamination problem.

### SUMMARY OF THE INVENTION

An object of the present invention provides an improved biological detection cartridge and a method for performing the same while simplifying the sample-injecting procedure and avoiding the contamination problem.

Another object of the present invention provides an improved biological detection cartridge and a method for performing the same for providing a good incubation environment and facilitating observing the incubation result.

A further object of the present invention provides an improved biological detection cartridge and a method for performing the same for effectively achieving quantitative injection and avoiding the sample-injecting error.

In accordance with an aspect of the present invention, a biological detection cartridge is provided. The biological detection cartridge includes a detection unit. The detection unit includes an incubation unit, a port, a ventilation structure and a cover. The incubation unit includes a culture well. The port is disposed adjacent to a first side of the incubation unit. The ventilation structure is disposed adjacent to a second side of the incubation unit. The ventilation structure includes a plurality of channels in communication with the incubation unit. The cover is disposed over the incubation unit and the ventilation structure.

In an embodiment, the ventilation structure includes a plurality of ribs discretely arranged at a spacing interval and defining the plurality of channels.

In an embodiment, the plurality of ribs of the ventilation structure are in close contact with the cover, and every two ribs of the plurality of ribs are separated by the spacing interval to define one of the plurality of channels. A diameter of the channel is in a range between 0.05 mm and 1 mm.

In an embodiment, the biological detection cartridge further includes a quantitative structure, which is disposed between the port and the incubation unit. The quantitative structure is covered by the cover. A gap is formed between the quantitative structure and the cover. The gap is in a range between 0.05 mm and 1 mm.

In an embodiment, the quantitative structure has a first surface, a second surface and a tip end. The first surface faces the incubation unit. The second surface faces the port. The tip end is located at a junction between the first surface and the second surface. A first angle is formed between the first surface and a horizontal plane passing through the tip end, and a second angle is formed between the second surface and the horizontal plane passing through the tip end. The second angle is greater than the first angle. The second angle is greater than (90−θc) degrees, wherein θc is a surface contact angle.

In an embodiment, the culture well is a tapered recess, and a diameter of the tapered recess from top to bottom is gradually decreased.

In an embodiment, a first end of the port is located away from the incubation unit, a second end of the port is located near the incubation unit, and the port has a slant surface. The slant surface is inclined downwardly from the first end of the port to the second end of the port at an inclination angle. The inclination angle of the slant surface is equal to or greater than 10 degrees.

In an embodiment, a first side of the ventilation structure is located near the incubation unit, a second side of the ventilation structure is located away from the incubation unit, and the biological detection cartridge further comprises a slot. The slot is disposed adjacent to the second side of the ventilation structure. The slot is in communication with the ventilation structure.

In accordance with another aspect of the present invention, a method for performing a biological detection cartridge includes the following steps. Firstly, a biological detection cartridge is provided. The biological detection cartridge includes a detection unit. The detection unit includes a port, an incubation unit, a ventilation structure, a quantitative structure and a cover. The incubation unit includes a culture well. The port is disposed adjacent to a first side of the incubation unit. The ventilation structure is disposed adjacent to a second side of the incubation unit. The ventilation structure includes a plurality of channels in communication with the incubation unit. The quantitative structure is disposed between the port and the incubation unit. The quantitative structure, the incubation unit and the ventilation structure are covered by the cover. Then, the biological detection cartridge is in an inclined placement state, and sample is added into the port. Consequently, the sample is transferred to the incubation unit through a gap between the quantitative structure and the cover and the sample is stopped in the plurality of channels. After the sample is filled in the incubation unit, the biological detection cartridge is in a horizontal placement state. Consequently, a surplus portion of the sample is stopped to remain in the port by the quantitative structure.

Preferably, when the biological detection cartridge is placed on an inclined jig tool, the biological detection cartridge is in the inclined placement state. In an embodiment, the jig tool includes an inclined surface, and an inclination angle of the inclined surface is in a range between 10 degrees and 80 degrees.

In an embodiment, the method further includes steps of covering the port with a gas permeable film, and incubating and observing the biological detection cartridge.

In an embodiment, the ventilation structure comprises a plurality of ribs, and every two ribs of the plurality of ribs are separated by a spacing interval to define one of the plurality of channels, wherein a diameter of the channel is in a range between 0.05 mm and 1 mm.

In an embodiment, the gap between the quantitative structure and the cover is in a range between 0.05 mm and 1 mm.

In an embodiment, the quantitative structure has a first surface, a second surface and a tip end. The first surface faces the incubation unit. The second surface faces the port. The tip end is located at a junction between the first surface and the second surface.

In an embodiment, a first angle is formed between the first surface and a horizontal plane passing through the tip end, and a second angle is formed between the second surface and the horizontal plane passing through the tip end. The second angle is greater than the first angle.

The above contents of the present invention will become more readily apparent to those ordinarily skilled in the art after reviewing the following detailed description and accompanying drawings, in which:

### BRIEF DESCRIPTION OF THE DRAWINGS

FIG. 1 schematically illustrates a 96-well plate for performing an antimicrobial susceptibility test;

FIG. 2 is a schematic perspective view illustrating a biological detection cartridge according to a first embodiment of the present invention;

FIG. 3 is a schematic exploded view illustrating the biological detection cartridge as shown in FIG. 2;

FIG. 4 is a schematic cross-sectional view illustrating the biological detection cartridge as shown in FIG. 2 and taken along the line AA;

FIG. 5 is a schematic enlarged view illustrating a portion of the biological detection cartridge as shown in FIG. 2;

FIG. 6A schematically illustrates the diameter of the channel of the ventilation structure and a divergence angle;

FIG. 6B schematically illustrates a contact angle between a liquid and a surface of a solid;

FIG. 7 is a schematic enlarged view illustrating the quantitative structure of the biological detection cartridge as shown in FIG. 2;

FIG. 8 is a schematic perspective view illustrating a biological detection cartridge according to a second embodiment of the present invention;

FIG. 9 schematically illustrates the biological detection cartridge in an inclined placement state;

FIG. 10 schematically illustrates an experiment of using the biological detection cartridge of the present invention to incubate a red blood cell dilution solution;

FIG. 11A schematically illustrates the result of using a 96-well plate to incubate and detect microorganisms; and

FIG. 11B schematically illustrates the result of using the biological detection cartridge of the present invention to incubate and detect microorganisms.

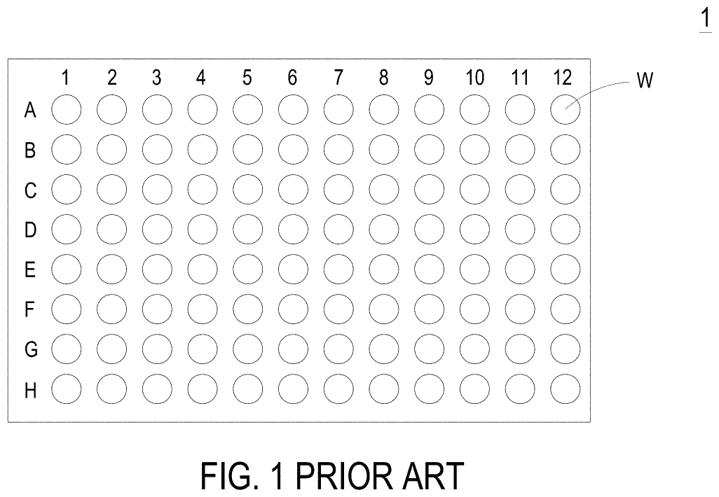

### DETAILED DESCRIPTION OF THE PREFERRED EMBODIMENT

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

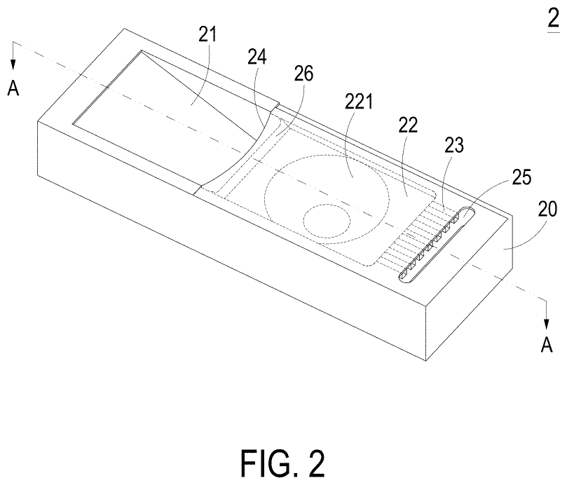

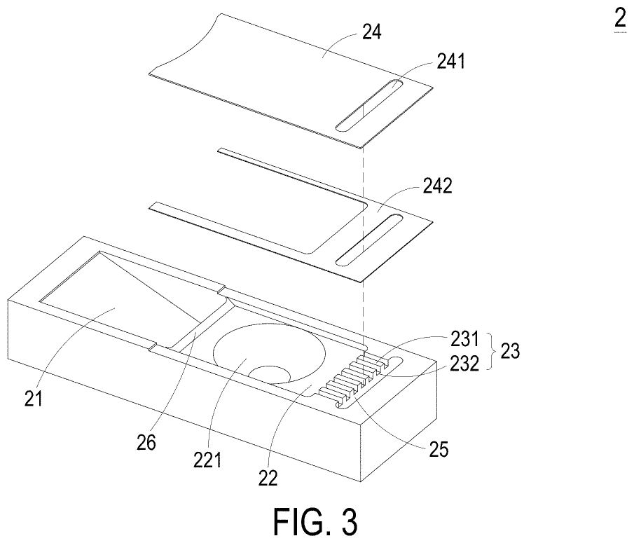

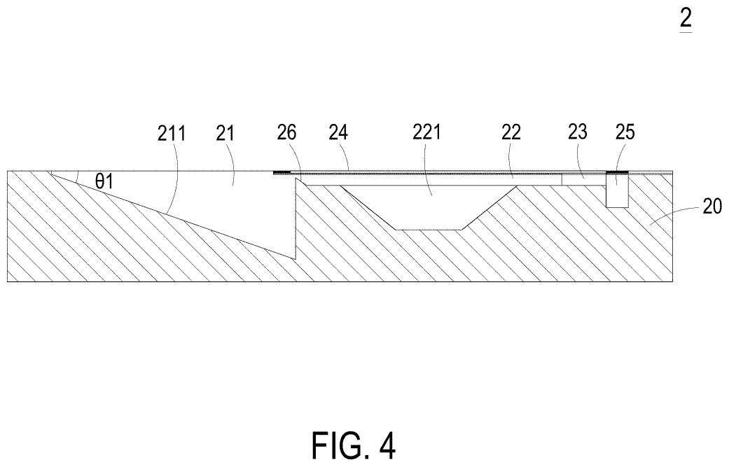

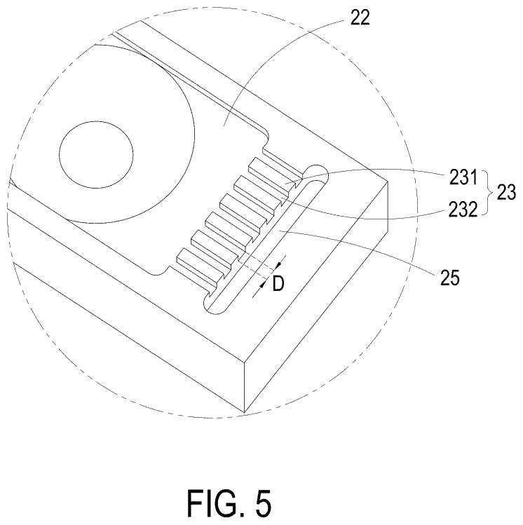

FIG. 2 is a schematic perspective view illustrating a biological detection cartridge according to a first embodiment of the present invention. FIG. 3 is a schematic exploded view illustrating the biological detection cartridge as shown in FIG. 2. FIG. 4 is a schematic cross-sectional view illustrating the biological detection cartridge as shown in FIG. 2 and taken along the line AA. FIG. 5 is a schematic enlarged view illustrating a portion of the biological detection cartridge as shown in FIG. 2. Please refer to FIGS. 2, 3, 4 and 5. The biological detection cartridge 2 comprises a detection unit 20. The detection unit 20 comprises a sample-injecting port 21, an incubation unit 22, a ventilation structure 23 and a cover 24.

The incubation unit 22 comprises a culture well 221 for incubating microorganisms (e.g., bacteria). The sample-injecting port 21 is disposed adjacent to a first side of the incubation unit 22. A sample can be added to the incubation unit 22 through the sample-injecting port 21. The ventilation structure 23 is disposed adjacent to a second side of the incubation unit 22. The first side and the second side of the incubation unit 22 are opposed to each other. The ventilation structure 23 comprises a plurality of ribs 231. The plurality of ribs 231 are discretely arranged at a spacing interval. Consequently, a plurality of channels 232 are defined by the plurality of ribs 231. The plurality of channels 232 are in communication with the incubation unit 22. The cover 24 is disposed over the incubation unit 22 and the ventilation structure 23 in order to avoid contamination of the foreign matters.

In an embodiment, the sample is an under-test bacteria solution. Moreover, an antibiotic agent is previously added to the culture well 221. Consequently, an antimicrobial susceptibility test can be performed on the microorganisms. Especially, after the antibiotic agent is added to the culture well 221, the antibiotic agent undergoes a drying process. Consequently, the dried antibiotic agent can be stored for the subsequent antimicrobial susceptibility test.

Preferably but not exclusively, the culture well 221 of the incubation unit 22 is a circular recess. The culture well 221 has a tapered structure. That is, the diameter of the culture well 221 from top to bottom is gradually decreased. Due to the tapered structure, colonies of the microorganisms (e.g., bacteria) can be centralized to a middle region of the culture well 221 in order to facilitate observation.

A first end of the sample-injecting port 21 is located away from the incubation unit 22. A second end of the sample-injecting port 21 is located near the incubation unit 22. In an embodiment, the sample-injecting port 21 has a slant surface 211. The slant surface 211 is inclined downwardly from the first end of the sample-injecting port 21 to the second end of the sample-injecting port 21 at an inclination angle θ1. The inclination angle θ1 is greater than 10 degrees. More preferably, the inclination angle θ1 is in the range between 30 degrees and 80 degrees. During the sample-injecting procedure, the biological detection cartridge 2 is in an inclined placement state. After the sample is added into the sample-injecting port 21, the sample is gradually collected at the bottom of the sample-injecting port 21 along the slant surface 211. When the liquid level of the sample is close to the cover 24, the sample flows into the incubation unit 22 through a vacant space between the incubation unit 22 and the cover 24. Consequently, the sample-injecting procedure can be simplified.

As mentioned above, the plurality of ribs 231 of the ventilation structure 23 are discretely arranged at a spacing interval. That is, a channel 232 is defined by every two adjacent ribs 231. In accordance with the present invention, the diameter of the channel 232 of the ventilation structure 23 is properly designed, and the plurality of ribs 231 of the ventilation structure 23 are in close contact with the cover 24 to be tightly covered by the cover 24. During the sample-injecting procedure, the channels 232 can be used as capillary vents, and thus the sample can be injected more smoothly. Moreover, the channels 232 can be used as liquid-stopping structures. That is, the sample is stopped in the channels 232, and the sample will not flow out of the channels 232. In such way, the problems of contamination and infection are avoided, and the safety protection efficacy is enhanced. During the incubating procedure, the oxygen gas can be transferred through the channels 232. Consequently, the sufficient amount of oxygen gas is provided to the incubation unit 22 for incubating the microorganisms.

A first side of the ventilation structure 23 is located near the incubation unit 22. A second side of the ventilation structure 23 is located away from the incubation unit 22. In an embodiment, the biological detection cartridge 2 further comprises a slot 25. The slot 25 is disposed adjacent to the second side of the ventilation structure 23. The slot 25 is in communication with the ventilation structure 23. That is, the slot 25 is in communication with the channels 232. The cover 24 has an opening 241 corresponding in position to the slot 25. Consequently, the slot 25 is exposed through the opening 241. During the sample-injecting procedure, the slot 25 is used as a vent. During the incubating procedure, the slot 25 is an input port for feeding the oxygen gas.

In another embodiment, the slot 25 of the biological detection cartridge 2 is omitted. Under this circumstance, the distal end of the ventilation structure 23 is the distal end of the detection unit 20, and the channels 232 of the ventilation structure 23 are in communication with the environment directly. During the sample-injecting procedure, the channels 232 are used as vents. During the incubating procedure, the channels 232 are input ports for feeding the oxygen gas.

As shown in FIG. 5, the channel 232 of the ventilation structure 23 has a diameter D. The diameter D of the channel 232 is determined according to the oxygen permeability, the liquid surface tension and the processing capability. In an embodiment, the diameter D of the channel 232 is in the range between 0.05 mm and 1 mm, and preferably 0.5 mm.

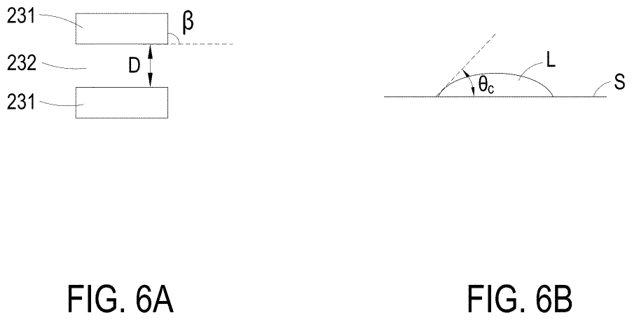

In addition to the diameter D of the channel 232, the designing parameters of the ventilation structure 23 also comprises a divergence angle β, a surface tension α and a contact angle θc. FIG. 6A schematically illustrates the diameter of the channel 232 of the ventilation structure 23 and the divergence angle β. FIG. 6B schematically illustrates a contact angle θc between a liquid L and a surface of a solid S. For example, the solid S is the surface of the channel 232, and the solid S is made of PET.

In a simulation method about the diameter of the channel 232 of the ventilation structure 23, the divergence angle β related to the capillary negative pressure is set as 90 degrees, the surface tension a related to the solution property is set as 72.8 mN/m, and the contact angle θc related to the material property is set as 72 degrees. The simulation method has the following results. If the diameter D of the channel 232 is 0.5 mm, the liquid sample can be effectively stopped in the channel 232 of the ventilation structure 23 and will not flow into the slot 25. In addition, the channel 232 can withstand the pressure of a 5 cm height of the liquid. If the diameter D of the channel 232 is 1 mm, the liquid sample can be transferred to the slot 25 through the channel 232 of the ventilation structure 23. In other words, if the diameter D of the channel 232 is specially designed, the liquid sample can be effectively stopped. Consequently, the ventilation structure 23 can be used as a liquid stopping structure.

It is noted that the designing parameters of the ventilation structure 23 may be varied according to the practical requirements. For example, the divergence angle β is not restricted to 90 degrees. In another embodiment, the divergence angle β is (90−θc) degrees. In an embodiment, the channel 232 is a groove structure with the uniform width. In another embodiment, the channel 232 is a groove structure with non-uniform diameter, wherein the width from top to bottom is gradually decreased.

In an embodiment, the biological detection cartridge 2 further comprises a quantitative structure 26. The quantitative structure 26 is disposed between the sample-injecting port 21 and the incubation unit 22.

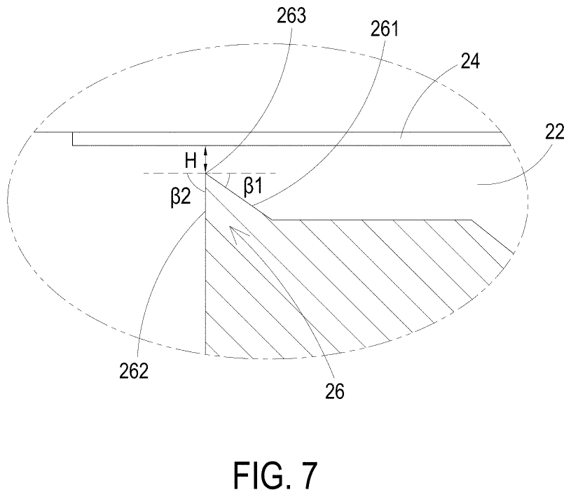

FIG. 7 is a schematic enlarged view illustrating the quantitative structure of the biological detection cartridge as shown in FIG. 2. Please refer to FIGS. 2, 3, 4 and 7. The quantitative structure 26 is covered by the cover 24. A gap H is formed between the quantitative structure 26 and the cover 24. During the sample-injecting procedure, the biological detection cartridge 2 is in an inclined placement state. After the sample is added into the sample-injecting port 21, the sample is transferred to the incubation unit 22 through the gap H between the quantitative structure 26 and the cover 24.

Please refer to FIG. 7 again. The quantitative structure 26 has a first surface 261, a second surface 262 and a tip end 263. The first surface 261 of the quantitative structure 26 faces the incubation unit 22. The second surface 262 of the quantitative structure 26 faces the sample-injecting port 21. The tip end 263 is located at the junction between the first surface 261 and the second surface 262 of the quantitative structure 26. An acute angle is formed between the surface 261 and the second surface 262 of the quantitative structure 26. The gap H is formed between the tip end 263 of the quantitative structure 26 and the cover 24. A first angle β1 is formed between the first surface 261 of the quantitative structure 26 and a horizontal plane passing through the tip end 263 of the quantitative structure 26. A second angle β2 is formed between the second surface 262 of the quantitative structure 26 and the horizontal plane passing through the tip end 263 of the quantitative structure 26. In an embodiment, β2 is greater than (90−θc) degrees, β2 is greater than β1, and H is equal to or less than 1 mm. Preferably but not exclusively, β2 is in the range between 80 degrees and 100 degrees, β1 is in the range between 20 degrees and 40 degrees, and H is in the range between 0.05 mm and 1 mm.

In a simulation method about the quantitative structure 26, the simulation parameters are set as: H=0.1 mm, β1=30 degrees, and β2=90 Degrees. The simulation result indicates that after the sample is added into the sample-injecting port 21, the sample can be transferred to the incubation unit 22 through the gap H between the quantitative structure 26 and the cover 24. Especially, when the liquid sample is returned back, the liquid sample is stopped at the tip end 263 of the quantitative structure 26. Consequently, the quantitative structure 26 is used as a one-way check valve to avoid the backflow of the liquid sample, and the quantitative function is achieved.

As mentioned above, during the sample-injecting procedure, the biological detection cartridge 2 is in the inclined placement state. After the sample is added into the sample-injecting port 21, the sample is transferred to the incubation unit 22 through the gap H between the quantitative structure 26 and the cover 24 and the sample is stopped in the channels 232 of the ventilation structure 23. After the sample is filled in the incubation unit 22, the biological detection cartridge 2 is in a horizontal placement state. Meanwhile, the surplus portion of the sample is stopped to remain in the sample-injecting port 21 by the tip end 263 of the quantitative structure 26 and the force of gravity, and the portion of the sample in the incubation unit 22 is not returned back to the sample-injecting port 21. Due to the quantitative structure 26, the volume of the sample added to the biological detection cartridge 2 for each time is constant. Consequently, the sample-injecting error of using the pipette to add the sample in the conventional technology will be avoided.

In the above embodiment, the first surface 261 and the second surface 262 of the quantitative structure 26 are flat surfaces. It is noted that the profiles of the first surface 261 and the second surface 262 of the quantitative structure 26 are not restricted. For example, in another embodiment, the first surface 261 and the second surface 262 of the quantitative structure 26 are curvy surfaces.

In an embodiment, the cover 24 is a transparent hydrophilic film or a translucent hydrophilic film. Moreover, the growth condition of the microorganisms in the culture well 221 can be observed by the operator directly.

In an embodiment, the cover 24 is adhered onto the top surface of the detection unit 20 through an adhesive layer 242. Consequently, the incubation unit 22, the ventilation structure 23 and the quantitative structure 26 are covered by the cover 24, and the slot 25 is exposed. Preferably but not exclusively, the adhesive layer 242 is a double-sided adhesive. It is noted that numerous modifications and alterations may be made while retaining the teachings of the invention. In another embodiment, the adhesive layer 242 is omitted, but the cover 24 is fixed on the detection unit 20 through a locking means.

In an embodiment, after the sample-injecting procedure is completed and the biological detection cartridge 2 is in the horizontal placement state, the sample-injecting port 21, the ventilation structure 23 and the slot 25 are covered by a gas permeable film (not shown). Consequently, the possibility of contaminating and vaporizing the sample will be minimized. Moreover, the gas permeable film is helpful for gas exchange. Consequently, the oxygen gas can be provided to the microorganisms in the incubation unit 22 through the gas permeable film and the gap H between the quantitative structure 26 and the cover 24. That is, the biological detection cartridge 2 provides a good incubation environment for the microorganisms. The oxygen gas can be transferred to the incubation unit 22 through the channels 232 of the ventilation structure 23 and the gap H between the quantitative structure 26 and the cover 24. Consequently, the sufficient oxygen gas can be provided to the microorganisms in the incubation unit 22.

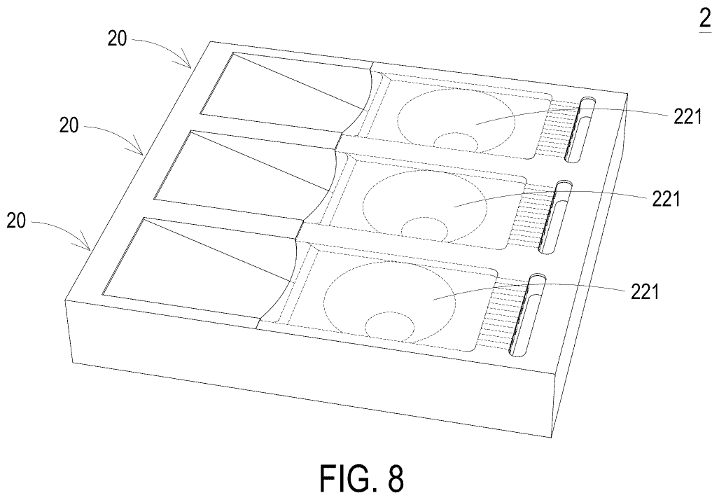

FIG. 8 is a schematic perspective view illustrating a biological detection cartridge according to a second embodiment of the present invention. In this embodiment, the biological detection cartridge 2 comprises a plurality of detection units 20. For example, as shown in FIG. 8, the biological detection cartridge 2 comprises three detection units 20. The structure of the detection unit 20 of this embodiment is identical to that of the detection unit 20 as shown in FIG. 2. In this embodiment, antibiotic agents with different amounts are previously added to the culture wells 221 of the plurality of incubation units 22. Consequently, an antimicrobial susceptibility test can be performed on the microorganisms. After the samples with the constant amount are added to the culture wells 221 of the plurality of incubation units 22, the antibiotic agents with different concentrations are contained in the culture wells 221. By observing the growth condition of the microorganisms (e.g. bacteria) in the culture wells 221 with different concentrations of antibiotic agents, the drug resistance of the microorganisms (e.g. bacteria) can be detected.

In another embodiment, the biological detection cartridge 2 comprises 12 detection units 20. One of the 12 detection units 20 is used as a positive control unit, one of the 12 detection units 20 is used as a negative control unit, and 10 of the 12 detection units 20 contain the antibiotic agents with different concentrations. Consequently, a plurality of antimicrobial susceptibility tests can be performed on the microorganisms (e.g. bacteria) according to different concentrations. In some embodiments, the 10 detection units 20 are divided into two groups, so that the antimicrobial susceptibility tests can be performed on different types of microorganisms. In some other embodiments, the 12 detection units 20 are divided into two groups, so that the antimicrobial susceptibility tests can be performed on the microorganisms (e.g. bacteria) according to different types of the antibiotic agents. It is noted that the number of the detection units 20 in the biological detection cartridge 2 may be varied according to the practical requirements.

The present invention further comprises a method for performing the biological detection cartridge 2. The method comprises a sample-injecting procedure and a quantitative procedure. The biological detecting method will be described as follows.

Firstly, a biological detection cartridge 2 with at least one detection unit 20 is provided. The detection unit 20 comprises a sample-injecting port 21, an incubation unit 22, a ventilation structure 23, a quantitative structure 26 and a cover 24. The incubation unit 22 comprises a culture well 221. The sample-injecting port 21 is disposed adjacent to a first side of the incubation unit 22. The ventilation structure 23 is disposed adjacent to a second side of the incubation unit 22. The ventilation structure 23 comprises a plurality of ribs 231. The plurality of ribs 231 are discretely arranged at a spacing interval. Consequently, a plurality of channels 232 are defined by the plurality of ribs 231. The plurality of channels 232 are in communication with the incubation unit 22. The quantitative structure 26 is disposed between the sample-injecting port 21 and the incubation unit 22. The quantitative structure 26, the incubation unit 22 and the ventilation structure 23 are covered by the cover 24.

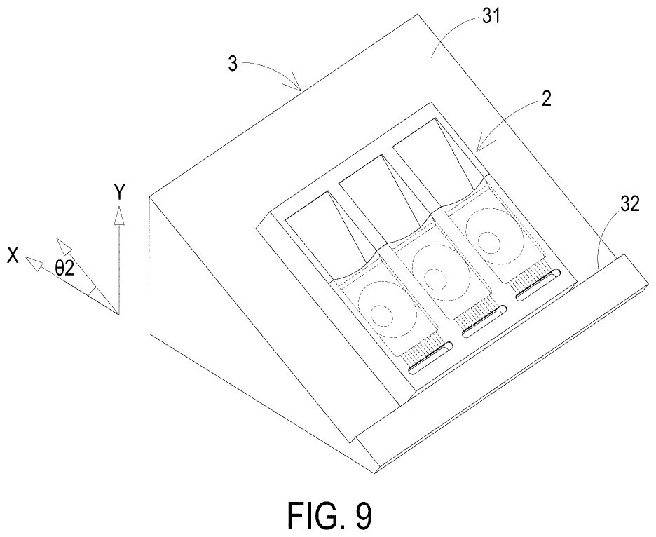

Then, the biological detection cartridge 2 is in an inclined placement state. FIG. 9 schematically illustrates the biological detection cartridge in an inclined placement state. By placing the biological detection cartridge 2 on an inclined jig tool 3, the biological detection cartridge 2 can be in an inclined placement state. The jig tool 3 comprises an inclined surface 31 and a support surface 32. The inclined surface 31 is inclined upwardly at an inclination angle θ2 with respect to a horizontal axis X. The inclination angle θ2 is equal to or greater than 10 degrees. For example, the inclination angle θ2 is in the range between 10 degrees and 80 degrees. The support surface 32 is located at a bottom edge of the inclined surface 31 and substantially perpendicular to the inclined surface 31. The support surface 32 is used for supporting and stopping the biological detection cartridge 2. After the biological detection cartridge 2 is placed on the inclined surface 31 and supported by the support surface 32, the biological detection cartridge 2 is in the inclined placement state. After the sample is added into the sample-injecting port 21, the sample is transferred to the incubation unit 22 through the gap H between the quantitative structure 26 and the cover 24 according to the force of gravity and the sample is stopped in the channels 232 of the ventilation structure 23.

After the sample is filled in the incubation unit 22, the biological detection cartridge 2 is removed from the jig tool 3 and placed on a horizontal operation plane. Consequently, the biological detection cartridge 2 is in a horizontal placement state. Meanwhile, the surplus portion of the sample is stopped to remain in the sample-injecting port 21 by the tip end 263 of the quantitative structure 26 and the force of gravity, and the portion of the sample in the incubation unit 22 is not returned back to the sample-injecting port 21. Due to the quantitative structure 26, the volume of the sample added to the biological detection cartridge 2 for each time is constant. After the sample-injecting procedure is completed and the biological detection cartridge 2 is in the horizontal placement state, the sample-injecting port 21, the ventilation structure 23 and the slot 25 are covered by a gas permeable film (not shown). Consequently, the possibility of contaminating and vaporizing the sample will be minimized. Then, the biological detection cartridge 2 is placed in an incubation box and incubated for a specified time period (e.g., 16 to 20 hours). Then, the incubation result is observed with naked eyes.

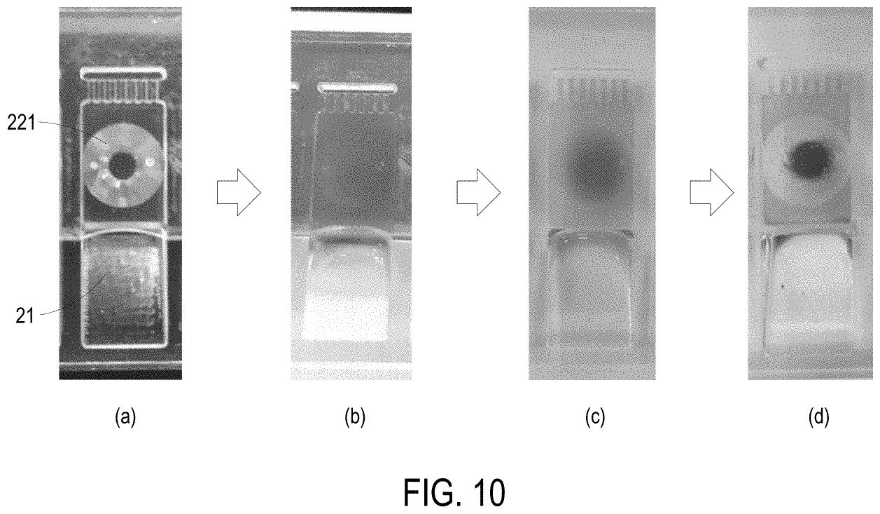

FIG. 10 schematically illustrates an experiment of using the biological detection cartridge of the present invention to incubate a red blood cell dilution solution. The red blood cell dilution solution is adjusted to have a blood volume ratio of 4%. Firstly, in a step (a), the biological detection cartridge 2 is in the inclined placement state. In a step (b), 120 μL of red blood cell dilution solution is added to the sample-injecting port 21. In a step (c), the biological detection cartridge 2 is in the horizontal placement state and covered by a gas permeable film. After the biological detection cartridge 2 is placed in an incubation box and incubated at 36° C. for 20 hours, the biological detection cartridge 2 is removed for observation (step (d)). As shown in the drawings, the red blood cells are settled in the culture well 221. According to the simulation result, it is found that the use of the biological detection cartridge 2 is helpful to inject sample at constant amount and observe the detection result.

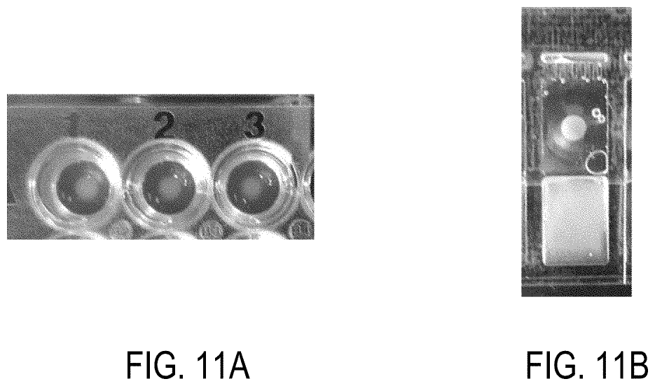

FIG. 11A schematically illustrates the result of using a 96-well plate to incubate and detect microorganisms. FIG. 11B schematically illustrates the result of using the biological detection cartridge of the present invention to incubate and detect microorganisms. The test strain is a common Escherichia coli (E. coli). Firstly, 10⁵ CFU/mL of bacteria are added to a liquid broth culture solution. Then, 100˜120 μL of bacteria-containing culture solution is added to a 96-well plate and the biological detection cartridge 2. Then, the 96-well plate is covered by a top lid, and the biological detection cartridge 2 is covered by a gas permeable film. After the 96-well plate and the biological detection cartridge 2 are placed in an incubation box and incubated at 36° C. for 20 hours, the 96-well plate and the biological detection cartridge 2 are removed for observation.

The incubation result of using the 96-well plate is shown in FIG. 11A. It is found that the bacteria settle to the bottom and form a circular colony. The incubation result of using the biological detection cartridge 2 is shown in FIG. 11B. Similarly, it is found that the bacteria settle to the bottom and form a circular colony. Consequently, the biological detection cartridge 2 is capable of incubating the microorganisms successfully, and the incubation result of the biological detection cartridge 2 and the incubation result of the 96-well plate are identical.

From the above descriptions, the present invention provides a biological detection cartridge with at least one detection unit. The detection unit comprises a sample-injecting port, an incubation unit, a ventilation structure and a cover. The sample-injecting port is disposed adjacent to a first side of the incubation unit. The ventilation structure is disposed adjacent to a second side of the incubation unit. The ventilation structure comprises a plurality of ribs. The plurality of ribs are discretely arranged at a spacing interval. Consequently, a plurality of channels are defined by the plurality of ribs. During the sample-injecting procedure, the channels can be used as capillary vents, and thus the sample can be injected more smoothly. Moreover, the channels can be used as liquid-stopping structures. That is, the sample is stopped in the channels, and the sample will not flow out of the channels. In such way, the problems of contamination and infection are avoided, and the safety protection efficacy is enhanced. During the incubating procedure, the oxygen gas can be transferred through the channels. Consequently, the sufficient amount of oxygen gas is provided to the incubation unit for incubating the microorganisms. The biological detection cartridge can provide a good incubation environment and facilitate the observation of the incubation result. The biological detection cartridge further comprises a quantitative structure. The present invention also provides a method for performing the biological detection cartridge, and the method comprising a sample-injecting procedure and a quantitative procedure. The biological detection cartridge and the method for performing the same are effective to inject the sample at constant amount and avoid the sample-injecting error. In case that the biological detection cartridge comprises a plurality of detection units, the antibiotic agents with different amounts are previously added to the culture wells of the plurality of incubation units. Consequently, an antimicrobial susceptibility test can be performed on the microorganisms.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A biological detection cartridge comprising a detection unit, the detection unit comprising:
an incubation unit comprising a culture well;
a port disposed adjacent to a first side of the incubation unit;
a ventilation structure disposed adjacent to a second side of the incubation unit, wherein the ventilation structure comprises a plurality of channels in communication with the incubation unit; and
a cover disposed over the incubation unit and the ventilation structure.

2. The biological detection cartridge according to claim 1, wherein the ventilation structure comprises a plurality of ribs discretely arranged at a spacing interval and defining the plurality of channels.

3. The biological detection cartridge according to claim 2, wherein the plurality of ribs are in close contact with the cover, and every two ribs of the plurality of ribs are separated by the spacing interval to define one of the plurality of channels, wherein a diameter of the channel is in a range between 0.05 mm and 1 mm.

4. The biological detection cartridge according to claim 1, wherein the biological detection cartridge further comprises a quantitative structure, which is disposed between the port and the incubation unit, wherein the quantitative structure is covered by the cover, and a gap is formed between the quantitative structure and the cover.

5. The biological detection cartridge according to claim 4, wherein the gap is in a range between 0.05 mm and 1 mm.

6. The biological detection cartridge according to claim 4, wherein the quantitative structure has a first surface, a second surface and a tip end, wherein the first surface faces the incubation unit, the second surface faces the port, and the tip end is located at a junction between the first surface and the second surface.

7. The biological detection cartridge according to claim 6, wherein a first angle is formed between the first surface and a horizontal plane passing through the tip end, and a second angle is formed between the second surface and the horizontal plane passing through the tip end, wherein the second angle is greater than the first angle.

8. The biological detection cartridge according to claim 7, wherein the second angle is greater than (90−θc) degrees, wherein θc is a surface contact angle.

9. The biological detection cartridge according to claim 1, wherein the culture well is a tapered recess, and a diameter of the tapered recess from top to bottom is gradually decreased.

10. The biological detection cartridge according to claim 1, wherein a first end of the port is located away from the incubation unit, a second end of the port is located near the incubation unit, and the port has a slant surface, wherein the slant surface is inclined downwardly from the first end of the port to the second end of the port at an inclination angle.

11. The biological detection cartridge according to claim 10, wherein the inclination angle of the slant surface is equal to or greater than 10 degrees.

12. The biological detection cartridge according to claim 1, wherein a first side of the ventilation structure is located near the incubation unit, a second side of the ventilation structure is located away from the incubation unit, and the biological detection cartridge further comprises a slot, wherein the slot is disposed adjacent to the second side of the ventilation structure, and the slot is in communication with the ventilation structure.

13. A method for performing a biological detection cartridge, comprising steps of:
providing a biological detection cartridge, wherein the biological detection cartridge comprises a detection unit, and the detection unit comprises a port, an incubation unit, a ventilation structure, a quantitative structure and a cover, wherein the incubation unit comprises a culture well, the port is disposed adjacent to a first side of the incubation unit, the ventilation structure is disposed adjacent to a second side of the incubation unit, the ventilation structure comprises a plurality of channels in communication with the incubation unit, the quantitative structure is disposed between the port and the incubation unit, and the quantitative structure, the incubation unit and the ventilation structure are covered by the cover;
allowing the biological detection cartridge to be in an inclined placement state, and adding a sample into the port, so that the sample is transferred to the incubation unit through a gap between the quantitative structure and the cover and the sample is stopped in the plurality of channels; and
allowing the biological detection cartridge to be in a horizontal placement state after the sample is filled in the incubation unit, so that a surplus portion of the sample is stopped to remain in the port by the quantitative structure.

14. The method according to claim 13, wherein when the biological detection cartridge is placed on an inclined jig tool, the biological detection cartridge is in the inclined placement state.

15. The method according to claim 14, wherein the jig tool comprises an inclined surface, and an inclination angle of the inclined surface is in a range between 10 degrees and 80 degrees.

16. The method according to claim 13, further comprising steps of:
covering the port with a gas permeable film; and
incubating and observing the biological detection cartridge.

17. The method according to claim 13, wherein the ventilation structure comprises a plurality of ribs, and every two ribs of the plurality of ribs are separated by a spacing interval to define one of the plurality of channels, wherein a diameter of the channel is in a range between 0.05 mm and 1 mm.

18. The method according to claim 13, wherein the gap between the quantitative structure and the cover is in a range between 0.05 mm and 1 mm.

19. The method according to claim 13, wherein the quantitative structure has a first surface, a second surface and a tip end, wherein the first surface faces the incubation unit, the second surface faces the port, and the tip end is located at a junction between the first surface and the second surface.

20. The method according to claim 19, wherein a first angle is formed between the first surface and a horizontal plane passing through the tip end, and a second angle is formed between the second surface and the horizontal plane passing through the tip end, wherein the second angle is greater than the first angle.
